(19) United States
(12) Patent Application Publication
(10) Pub. No.: US 2024/0269464 A1
(43) Pub. Date: Aug. 15, 2024

(54) Sequentially Positioning Sets of Electrode Arrays at Nonoverlapping Positions to Ameliorate Skin Irritation During Tumor Treating Fields (TTFields) Therapy

(71) Applicant: Novocure GmbH, Root D4 (CH)

(72) Inventors: Avishai NAHSHONI, Haifa (IL); Oshrit Zeevi, Haifa (IL); Nadav Shapira, Haifa (IL)

(73) Assignee: Novocure GmbH, Root D4 (CH)

(21) Appl. No.: 18/435,375

(22) Filed: Feb. 7, 2024

Related U.S. Application Data
(60) Provisional application No. 63/444,725, filed on Feb. 10, 2023.

(51) Int. Cl. A61N 1/36; A61N 1/04

(52) U.S. Cl. CPC A61N 1/36002; A61N 1/0476; A61N 1/36034

(57) ABSTRACT

Alternating electric fields can be applied to a subject's body by (a) applying alternating voltages between electrode elements positioned on four primary non-overlapping regions on the subject's body during a first interval of time; and (b) applying alternating voltages between other electrode elements positioned on four secondary non-overlapping regions on the subject's body during a second interval of time. None of the secondary regions overlap any of the primary regions, and the first and second intervals of time are nonoverlapping. Due to the nonoverlapping nature in both space and time, the incidence of skin irritation will be reduced. And to the extent that skin irritation does occur during a given interval of time, it will have an opportunity to heal during the subsequent interval of time.

### CROSS REFERENCE TO RELATED APPLICATIONS

This application claims the benefit of US Provisional Application 63/444,725, filed Feb. 10, 2023, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 150-250 kHz). In the prior art Optune® system, TTFields are delivered to patients via four transducer arrays that are placed on the patient's skin near the tumor. The transducer arrays are arranged in two pairs, with one pair of transducer arrays positioned to the left and right of the tumor, and the other pair of transducer arrays positioned anterior and posterior to the tumor. Each transducer array is connected via a multi-wire cable to an AC signal generator. The AC signal generator (a) sends an AC current through the anterior/posterior (A/P) pair of transducer arrays for 1 second, which induces an electric field with a first direction through the tumor; then (b) sends an AC current through the left/right (L/R) pair of arrays for 1 second, which induces an electric field with a second direction through the tumor; then repeats steps (a) and (b) for the duration of the treatment. Each transducer array includes a plurality (e.g., between 9 and 30) of electrode elements.

Alternating electric fields can also be used to treat medical conditions other than tumors. For example, as described in U.S. Pat. No. 10,967,167 (which is incorporated herein by reference in its entirety), alternating electric fields can be used to increase the permeability of the blood brain barrier so that, e.g., chemotherapy drugs can reach the brain.

When the Optune® system is used to treat a tumor using TTFields, the transducer arrays are affixed to the subject's skin at positions that are selected to provide the strongest electric field within the tumor. The transducer arrays are affixed to the subject's body using self-adhesive backings, and they remain affixed at the same position for an extended period of time (e.g., 3-4 days). After this extended period of time, the transducer arrays are removed so that the subject's skin can be shaved and bathed. A fresh set of transducer arrays are then affixed to the subject's body at the same general location as before, shifted by less than one inch from the previous position. (See the Optune Patient Information and Operation Manual, Document Number: QSD-QR-704. REV 05, January 2019.) Treatment using Optune® typically continues for many months or even years, and the transducer arrays are replaced every 3-4 days.

Some patients experience skin irritation or itchiness beneath the transducer arrays. And while these conditions can usually be treated with a steroid cream, a subset of these patients may find the irritation or itchiness to be very disagreeable.

### SUMMARY OF THE INVENTION

Unlike the prior art approach in which successive sets of transducer arrays are all placed on the subject's body at the same general locations (i.e., a set of four locations that have been selected to provide the strongest electric field within the tumor), this application describes a number of alternative approaches to positioning successive sets of transducer arrays on the subject's body at different locations (e.g., every 3-4 days). More specifically, in some embodiments, successive sets of transducer arrays are placed on the subject's body at positions that are completely non-overlapping. In other embodiments, successive sets of transducer arrays are placed on the subject's body at positions that overlap by not more than 20%. And although none of those positionings may deliver the absolute strongest electric field to the tumor, each of the positionings will provide an electric field that is strong enough to effectively treat the tumor. And notably, because successive sets of transducer arrays are placed on different positions on the subject's body (e.g., every 3-4 days), the subject will experience much less skin irritation. Moreover, to the extent that a portion of the subject's skin does become irritated during a given 3-4 day interval, that portion of skin will receive an opportunity to heal during the next 3-4 day interval of time (during which it will not be covered by a transducer array).

One aspect of the invention is directed to a first method of applying alternating electric fields to a target region in a subject's body. The body has an anterior surface, a posterior surface, a left surface, and a right surface. The first method comprises (a) applying an alternating voltage at a frequency between 50 kHz and 1 MHz between a first set of one or more electrode elements positioned on a first region of the anterior surface and a second set of one or more electrode elements positioned on a second region of the posterior surface during a plurality of first portions of a first interval of time, and applying an alternating voltage at a frequency between 50 kHz and 1 MHz between a third set of one or more electrode elements positioned on a third region of the left surface and a fourth set of one or more electrode elements positioned on a fourth region of the right surface during a plurality of second portions of the first interval of time. The first method also comprises (b) applying an alternating voltage at a frequency between 50 kHz and 1 MHz between a fifth set of one or more electrode elements positioned on a fifth region of the anterior surface and a sixth set of one or more electrode elements positioned on a sixth region of the posterior surface during a plurality of first portions of a second interval of time, and applying an alternating voltage at a frequency between 50 kHz and 1 MHz between a seventh set of one or more electrode elements positioned on a seventh region of the left surface and a eighth set of one or more electrode elements positioned on a eighth region of the right surface during a plurality of second portions of the second interval of time. The first interval of time and the second interval of time are nonoverlapping. Less than 20% of the fifth region overlaps the first region, less than 20% of the sixth region overlaps the second region, less than 20% of the seventh region overlaps the third region, and less than 20% of the eighth region overlaps the fourth region.

In some instances of the first method, no part of the fifth region overlaps the first region, no part of the sixth region overlaps the second region, no part of the seventh region overlaps the third region, and no part of the eighth region overlaps the fourth region.

Some instances of the first method further comprise repeating steps (a) and (b) in an alternating sequence at least ten times.

In some instances of the first method, the target region comprises a tumor that has a centroid. And a ray projected directly anterior from the centroid does not intersect the first region, a ray projected directly posterior from the centroid does not intersect the second region, a ray projected directly anterior from the centroid does not intersect the fifth region, and a ray projected directly posterior from the centroid does not intersect the sixth region.

In some instances of the first method, the target region comprises a tumor that has a centroid. And a ray projected directly anterior from the centroid does not intersect at least one of the first region and the fifth region, a ray projected directly posterior from the centroid does not intersect at least one of the second region and the sixth region, a ray projected directly to the left from the centroid does not intersect at least one of the third region and the seventh region, and a ray projected directly to the right from the centroid does not intersect at least one of the fourth region and the eighth region.

In some instances of the first method, the second interval of time is separated from the first interval of time by less than 48 hours. In some instances of the first method, the first interval of time is at least 12 hours and the second interval of time is at least 12 hours. In some instances of the first method, each of the alternating voltages has a sinusoidal waveform. In some instances of the first method, each of the first, second, third, fourth, fifth, sixth, seventh, and eighth regions are located on the subject's abdomen.

Some instances of the first method further comprise (c) prior to the first interval of time, positioning the first, second, third, and fourth sets of one or more electrode elements on the first, second, third, and fourth regions, respectively; (d) after the first interval of time, removing the first, second, third, and fourth sets of one or more electrode elements from the subject's body; (e) prior to the second interval of time, positioning the fifth, sixth, seventh, and eighth sets of one or more electrode elements on the fifth, sixth, seventh, and eighth regions, respectively; and (f) after the second interval of time, removing the fifth, sixth, seventh, and eighth sets of one or more electrode elements from the subject's body.

Optionally, the instances described in the previous paragraph may further comprise comprising repeating steps (c), (a), (d), (e), (b), and (f), in that order, at least ten times. Optionally, in these instances, each of the first, second, third, fourth, fifth, sixth, seventh, and eighth regions may be located on the subject's abdomen.

Another aspect of the invention is directed to a second method of applying alternating electric fields to a tumor in a subject's body. The second method comprises (a) applying alternating voltages at a frequency between 50 kHz and 1 MHz between four primary sets of one or more electrode elements positioned on four primary non-overlapping regions on the subject's body during a first interval of time. And the second method also comprises (b) applying alternating voltages at a frequency between 50 kHz and 1 MHz between four secondary sets of one or more electrode elements positioned on four secondary non-overlapping regions on the subject's body during a second interval of time. Each of the primary regions has an area, and 0-20% of the area of each of the primary regions, taken alone, is overlapped by all of the secondary regions taken together. The first interval of time and the second interval of time are non-overlapping.

In some instances of the second method, none of the secondary regions overlap any of the primary regions.

Some instances of the second method further comprise repeating steps (a) and (b) in an alternating sequence at least ten times.

In some instances of the second method, the tumor has a centroid, a ray projected directly anterior from the centroid misses all of the primary regions and all of the secondary regions, and a ray projected directly posterior from the centroid misses all of the primary regions and all of the secondary regions.

In some instances of the second method, the second interval of time is separated from the first interval of time by less than 48 hours. In some instances of the second method, the first interval of time is at least 12 hours and the second interval of time is at least 12 hours. In some instances of the second method, each of the alternating voltages has a sinusoidal waveform. In some instances of the second method, each of the primary regions and each of the secondary regions are located on the subject's abdomen.

Some instances of the second method further comprise (c) prior to the first interval of time, positioning the primary set of one or more electrode elements on the primary regions; (d) after the first interval of time, removing the primary set of one or more electrode elements from the subject's body; (e) prior to the second interval of time, positioning the secondary set of one or more electrode elements on the secondary regions; and (f) after the second interval of time, removing the secondary set of one or more electrode elements from the subject's body.

Optionally, the instances described in the previous paragraph may further comprise repeating steps (c), (a), (d), (e), (b), and (f), in that order, at least ten times. Optionally, in these instances, each of the primary regions and each of the secondary regions are located on the subject's abdomen.

### BRIEF DESCRIPTION OF THE DRAWINGS

FIG. 1 depicts an example of four different views of a spherical target region T that includes a tumor in a subject's abdomen.

FIG. 2 depicts the regions on a subject's body where transducer arrays would conventionally be positioned in order to treat a tumor at the target region T using TTFields.

FIGS. 3A and 3B depict an alternative approach for positioning different sets of transducer arrays on a subject's body at different positions during different intervals of time to treat a tumor at the target region T using TTFields.

Various embodiments are described in detail below with reference to the accompanying drawings, wherein like reference numerals represent like elements.

### DESCRIPTION OF THE PREFERRED EMBODIMENTS

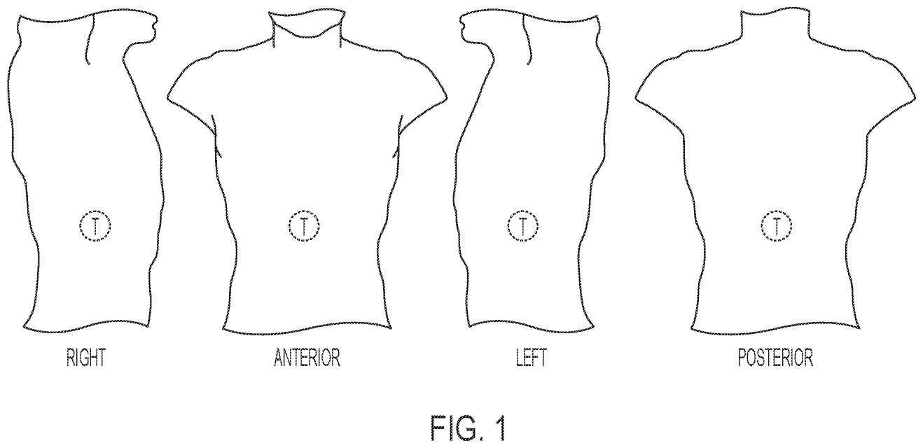

FIG. 1 depicts one example of four different views (i.e., right, anterior, left, and posterior views) of a spherical target region T that includes a tumor in a subject's abdomen.

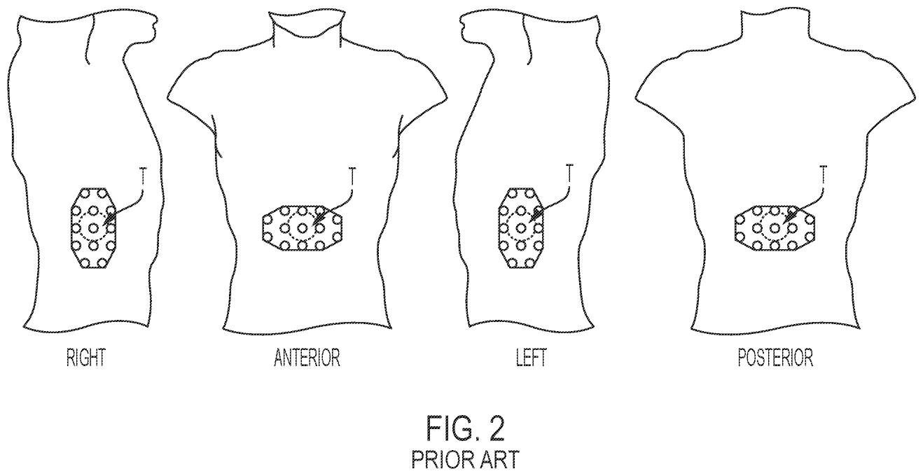

FIG. 2 depicts the regions on the right, front, left, and back of a subject's body where transducer arrays would conventionally be positioned in order to treat a tumor at the target region T using TTFields. One conventional approach for positioning the transducer arrays was to position the centroid of the transducer arrays directly to the right, front, left, and back of the centroid of the target region T, as depicted in FIG. 2. This approach is referred to herein as the dead-reckoning approach. Another conventional approach was to use specialized software (e.g., Novotal™ software) designed to recommend a set of positions for the transducer arrays that will maximize the strength of the electric field in the target region.

Notably, the recommendations generated by such software only deviated from the dead-reckoning locations depicted in FIG. 2 by a relatively small amount (e.g., less than 5 cm). In view of this relatively small shift in location, when either of the two conventional approaches was used, the positioning was such that (1) a ray projected directly anterior from the centroid of the tumor T would intersect the region where the anterior array is positioned; and (2) a ray projected directly posterior from the centroid of the tumor T would intersect the region where the posterior array is positioned.

Moreover, as explained above, each time a fresh set of transducer arrays was affixed to the subject's body, the set of transducer arrays was affixed at the same general locations, shifted by less than one inch from the baseline position. Thus, every time a fresh set of transducer arrays was affixed, the positioning was still such that (1) a ray projected directly anterior from the centroid of the tumor T would intersect the region where the anterior array is positioned; and (2) a ray projected directly posterior from the centroid of the tumor T would intersect the region where the posterior array is positioned.

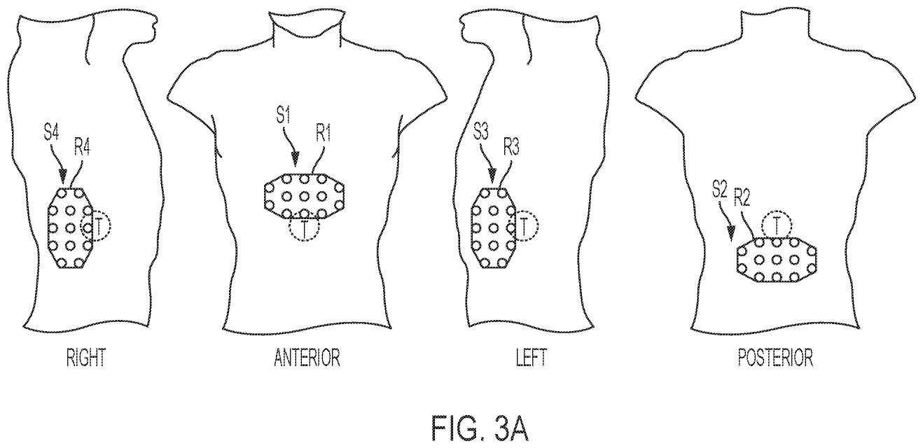

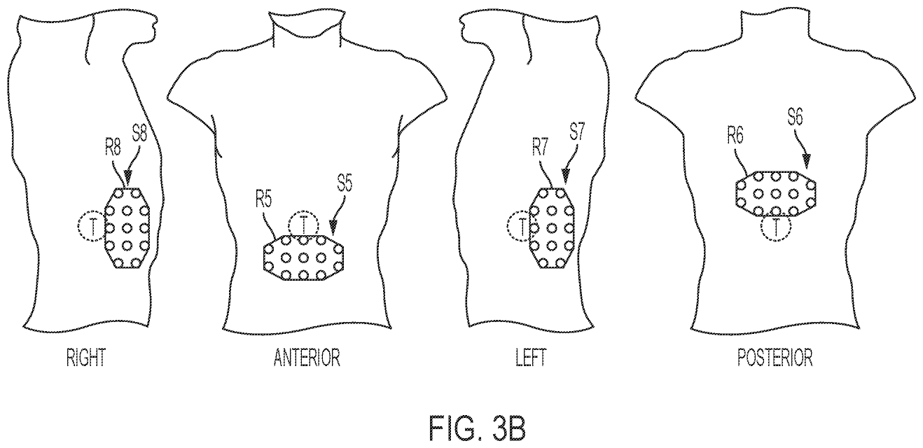

FIGS. 3A and 3B depict an alternative approach for positioning different sets of transducer arrays on a subject's body at different positions during different intervals of time. In this approach, a first set of four transducer arrays S1-S4 are positioned on respective regions R1-R4 of an anterior surface, a posterior surface, a left surface, and a right surface of a subject's body during a first interval of time (as depicted in FIG. 3A). And a second set of four transducer arrays S5-S8 are positioned on respective regions R5-R8 of the anterior, posterior, left, and right surfaces of the subject's body during a second interval of time (as depicted in FIG. 3B). The first interval of time and the second interval of time are nonoverlapping. Each of the transducer arrays S1-S8 includes one or more electrode elements. Because the transducer arrays are positioned at different positions during different intervals of time, skin irritation will be dramatically reduced, and any given patch of skin will have an opportunity to recover during the alternating intervals of time when it is not covered by a transducer array.

The positioning of the transducer arrays can be alternated between the positions depicted in FIG. 3A and the positions depicted in FIG. 3B e.g., every 3-4 days. For example, a first set of transducer arrays S1-S4 can be positioned on regions R1-R4 as depicted in FIG. 3A on days 1-3 of the treatment. During this three day first interval of time, the transducer arrays S1-S4 are used to apply TTFields to the subject's body as described below. At the end of day #3, the first set of transducer arrays S1-S4 is removed and discarded. A second set of transducer arrays S5-S8 is then positioned on regions R5-R8 as depicted in FIG. 3B on days 4-6 of the treatment. During this three day second interval of time, the transducer arrays S5-S8 are used to apply TTFields to the subject's body as described below. At the end of day #6, the second set of transducer arrays S5-S8 is removed and discarded.

A new first set of transducer arrays S1-S4 is then positioned on regions R1-R4 on days 7-9 of the treatment. During this three day first interval of time, the transducer arrays S1-S4 are used to apply TTFields to the subject's body. At the end of day #9, the set of transducer arrays S1-S4 is removed and discarded. A new second set of transducer arrays S5-S8 is then positioned on regions R5-R8 on days 10-12 of the treatment. During this three day second interval of time, the transducer arrays S5-S8 are used to apply TTFields to the subject's body. At the end of day 12, the set of transducer arrays S5-S8 is removed and discarded. This pattern in which the placement of the transducer arrays alternates every three days between regions R1-R4 during a first interval of time and regions R5-R8 during a second interval of time repeats continuously for the duration of the treatment. For example, if the duration of treatment is 60 days, the steps of (a) positioning the transducer arrays S1-S4 on regions R1-R4 during a first interval of time followed by (b) positioning the transducer arrays S5-S8 on regions R5-R8 during a second interval of time will repeat 10 times in an alternating sequence. If the duration of treatment is longer than 60 days, these steps (a) and (b) will repeat more than 10 times.

In some embodiments, no part of the fifth region R5 overlaps the first region R1, no part of the sixth region R6 overlaps the second region R2, no part of the seventh region R7 overlaps the third region R3, and no part of the eighth region R8 overlaps the fourth region R4 (e.g., as depicted in FIGS. 3A and 3B). In other embodiments (e.g., when the anatomy of a particular subject does not support a completely non-overlapping positioning), there may be a small amount of overlap. In these embodiments, less than 20% of the fifth region R5 overlaps the first region R1, less than 20% of the sixth region R6 overlaps the second region R2, less than 20% of the seventh region R7 overlaps the third region R3, and less than 20% of the eighth region R8 overlaps the fourth region R4.

During each of the first intervals of time, the direction of the electric field alternates between the anterior/posterior direction and the left/right direction. This may be accomplished by applying an alternating voltage at a frequency between 50 kHz and 1 MHz (e.g., 100 kHz-300 kHz) between the first transducer arrays S1 positioned on the first region R1 and the second transducer array S2 positioned on the second region R2 during a plurality of first portions of the first interval of time, and applying an alternating voltage at a similar frequency between the third transducer array S3 positioned on the third region R3 and a fourth transducer array S4 positioned on the fourth region R4 during a plurality of second portions of the first interval of time. In some embodiments, (a) an alternating voltage is applied between the first and second transducer arrays S1, S2 for e.g., one second, then (b) an alternating voltage is applied between the third and fourth transducer arrays S3, S4 for e.g., one second; and this two step sequence (a), (b) is repeated until the transducer arrays S1-S4 are removed.

Similarly, during each of the second intervals of time, the direction of the electric field alternates between the anterior/posterior direction and the left/right direction. This may be accomplished by applying an alternating voltage at a frequency between 50 kHz and 1 MHz (e.g., 100 kHz-300 kHz) between the fifth transducer arrays S5 positioned on the fifth region R5 and the sixth transducer array S6 positioned on the sixth region R6 during a plurality of first portions of the second interval of time, and applying an alternating voltage at a similar frequency between the seventh transducer array S7 positioned on the seventh region R7 and an eighth transducer array S8 positioned on the eighth region R8 during a plurality of second portions of the second interval of time. In some embodiments, (a) an alternating voltage is applied between the fifth and sixth transducer arrays S5, S6 for e.g., one second, then (b) an alternating voltage is applied between the seventh and eighth transducer arrays S7, S8 for e.g., one second; and this two step sequence (a), (b) is repeated until the transducer arrays S5-S8 are removed.

Unlike the prior art situation described above in connection with FIG. 2, when the transducer arrays S1-S4 are positioned at regions R1-R4 (as depicted in FIG. 3A), a ray projected directly anterior from the centroid of the tumor T will not intersect the first region R1, and a ray projected directly posterior from the centroid of the tumor T will not intersect the second region R2. Furthermore, when the transducer arrays S5-S6 are positioned at regions R5-R6 (as depicted in FIG. 3B), a ray projected directly anterior from the centroid of the tumor T will not intersect the fifth region R5, and a ray projected directly posterior from the centroid of the tumor T will not intersect the sixth region R6.

The strict positioning parameters described in the previous paragraph are not mandatory. To the contrary, in some embodiments some of the positionings are such that rays projected directly anterior or posterior from the centroid of the tumor T will intersect some, but not all of the regions. For example, in some embodiments, a ray projected directly anterior from the centroid of the tumor T does not intersect at least one of the first region R1 and the fifth region R5; a ray projected directly posterior from the centroid of the tumor T does not intersect at least one of the second region R2 and the sixth region R6; a ray projected directly to the left from the centroid of the tumor T does not intersect at least one of the third region R3 and the seventh region R7; and a ray projected directly to the right from the centroid of the tumor T does not intersect at least one of the fourth region R4 and the eighth region R8.

Notably, even though not all of the transducer arrays are positioned directly anterior or posterior from the centroid of the tumor T, simulations show that (a) the positioning depicted in FIG. 3A can yield an electric field of >1 V/cm in the target region that is strong enough to effectively treat the tumor and (b) the positioning depicted in FIG. 3B can also yield an electric field of >1 V/cm in the target region that is strong enough to effectively treat the tumor. Because the positioning of the transducer arrays alternates between the positioning depicted in FIG. 3A and the positioning depicted in FIG. 3B, the subject will always receive an electric field that is strong enough to effectively treat the tumor. In addition, skin irritation will be reduced because in some embodiments, no portion of skin is ever covered for more than three days at a time, and to the extent that skin irritation does occur, the skin will have a chance to heal during the three-day intervals of time when it remains uncovered. In other embodiments, the portion of skin that will be covered for more than three days at a time is dramatically reduced, which dramatically reduces the level of skin irritation with respect to the prior art positioning depicted in FIG. 2.

Because TTFields are more effective when the gaps in treatment are minimized, the gaps between the end of each first interval of time and the beginning of the subsequent second interval of time are preferably less than 96 hours, more preferably less than 48 hours, and still more preferably less than 24 hours.

In some preferred embodiments, each of the alternating voltages described above has a sinusoidal waveform. But in other embodiments, alternative waveforms including but not limited to square waves, triangle waves, etc. may be used.

In some preferred embodiments, each of the first, second, third, fourth, fifth, sixth, seventh, and eighth regions R1-R8 are located on the subject's abdomen.

Although the first interval of time is 3 days and the second interval of time is 3 days in the examples provided above, the duration of these intervals can vary. For example, each of these intervals could be at least 12 hours, at least 24 hours, or at least 48 hours.

In the examples described above, the first, second, third, and fourth regions are positioned on the anterior, posterior, left, and right surfaces of the subject's body, respectively; and the fifth, sixth, seventh, and eighth regions are positioned on the anterior, posterior, left, and right surfaces of the subject's body, respectively. But the concepts described above are not limited to the specific directions (i.e., anterior, posterior, left, and right) that appear in the examples described above. For example, all of the sets of electrode elements depicted in FIGS. 3A and 3B could be rotated by 45° about the longitudinal axis of the subject's body, and the tumor T would still be treatable using TTFields in a manner that is similar to the example described above in connection with FIGS. 3A and 3B.

Thus, the example described above in connection with FIGS. 3A and 3B can be generalized to a situation in which (a) alternating voltages at a frequency between 50 kHz and 1 MHz are applied between four primary sets of one or more electrode elements (i.e., between transducer arrays S1 and S2, and between transducer arrays S3 and S4) that are positioned on four primary non-overlapping regions on the subject's body (i.e., regions R1-R4) during a first interval of time; and (b) alternating voltages at a frequency between 50 kHz and 1 MHz are applied between four secondary sets of one or more electrode elements (i.e., between transducer arrays S5 and S6, and between transducer arrays S7 and S8) positioned on four secondary non-overlapping regions on the subject's body (i.e., regions R5-R8) during a second interval of time. Each of the primary regions R1-R4 has an area, and 0-20% of the area of each of the primary regions R1-R4, taken alone, is overlapped by all of the secondary regions R5-R6 taken together. The first interval of time and the second interval of time are nonoverlapping. In some embodiments (including the example depicted in FIGS. 3A and 3B), none of the secondary regions overlap any of the primary regions.

Finally, it is important to note that the usage of the identifiers (a), (b), (c), (d), etc. in the claims below does not imply a particular sequence in time for the corresponding steps. For while it is certainly possible that step (a) will precede step (b) in time, different sequencings of those steps are also possible, except in cases where a particular sequencing is inconsistent with the internal language of the various steps or with other language in the claims. For example, a step labeled (b) could precede a step labeled (a) in time. It is also possible for two or more steps to occur simultaneously or to overlap to an extent, except in cases where simultaneity or overlapping would be inconsistent with the internal language of the various steps or with other language in the claims.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of applying alternating electric fields to a target region in a subject's body, the body having an anterior surface, a posterior surface, a left surface, and a right surface, the method comprising:
(a) applying an alternating voltage at a frequency between 50 kHz and 1 MHz between a first set of one or more electrode elements positioned on a first region of the anterior surface and a second set of one or more electrode elements positioned on a second region of the posterior surface during a plurality of first portions of a first interval of time, and applying an alternating voltage at a frequency between 50 kHz and 1 MHz between a third set of one or more electrode elements positioned on a third region of the left surface and a fourth set of one or more electrode elements positioned on a fourth region of the right surface during a plurality of second portions of the first interval of time; and
(b) applying an alternating voltage at a frequency between 50 kHz and 1 MHz between a fifth set of one or more electrode elements positioned on a fifth region of the anterior surface and a sixth set of one or more electrode elements positioned on a sixth region of the posterior surface during a plurality of first portions of a second interval of time, and applying an alternating voltage at a frequency between 50 kHz and 1 MHz between a seventh set of one or more electrode elements positioned on a seventh region of the left surface and a eighth set of one or more electrode elements positioned on a eighth region of the right surface during a plurality of second portions of the second interval of time,
wherein the first interval of time and the second interval of time are non-overlapping,
wherein less than 20% of the fifth region overlaps the first region,
wherein less than 20% of the sixth region overlaps the second region,
wherein less than 20% of the seventh region overlaps the third region, and
wherein less than 20% of the eighth region overlaps the fourth region.

2. The method of claim 1,
wherein no part of the fifth region overlaps the first region,
wherein no part of the sixth region overlaps the second region,
wherein no part of the seventh region overlaps the third region,
wherein no part of the eighth region overlaps the fourth region.

3. The method of claim 1, further comprising repeating steps (a) and (b) in an alternating sequence at least ten times.

4. The method of claim 1, wherein the target region comprises a tumor, the tumor having a centroid,
wherein a ray projected directly anterior from the centroid does not intersect the first region,
wherein a ray projected directly posterior from the centroid does not intersect the second region,
wherein a ray projected directly anterior from the centroid does not intersect the fifth region, and
wherein a ray projected directly posterior from the centroid does not intersect the sixth region.

5. The method of claim 1, wherein the target region comprises a tumor, the tumor having a centroid,
wherein a ray projected directly anterior from the centroid does not intersect at least one of the first region and the fifth region,
wherein a ray projected directly posterior from the centroid does not intersect at least one of the second region and the sixth region,
wherein a ray projected directly to the left from the centroid does not intersect at least one of the third region and the seventh region, and
wherein a ray projected directly to the right from the centroid does not intersect at least one of the fourth region and the eighth region.

6. The method of claim 1, wherein the second interval of time is separated from the first interval of time by less than 48 hours.

7. The method of claim 1, wherein the first interval of time is at least 12 hours and the second interval of time is at least 12 hours.

8. The method of claim 1, wherein each of the alternating voltages has a sinusoidal waveform.

9. The method of claim 1, wherein each of the first, second, third, fourth, fifth, sixth, seventh, and eighth regions are located on the subject's abdomen.

10. The method of claim 1, further comprising:
(c) prior to the first interval of time, positioning the first, second, third, and fourth sets of one or more electrode elements on the first, second, third, and fourth regions, respectively;
(d) after the first interval of time, removing the first, second, third, and fourth sets of one or more electrode elements from the subject's body;
(e) prior to the second interval of time, positioning the fifth, sixth, seventh, and eighth sets of one or more electrode elements on the fifth, sixth, seventh, and eighth regions, respectively; and
(f) after the second interval of time, removing the fifth, sixth, seventh, and eighth sets of one or more electrode elements from the subject's body.

11. The method of claim 10, further comprising repeating steps (c), (a), (d), (e), (b), and (f), in that order, at least ten times.

12. The method of claim 11, wherein each of the first, second, third, fourth, fifth, sixth, seventh, and eighth regions are located on the subject's abdomen.

13. A method of applying alternating electric fields to a tumor in a subject's body, the method comprising:
(a) applying alternating voltages at a frequency between 50 kHz and 1 MHz between four primary sets of one or more electrode elements positioned on four primary non-overlapping regions on the subject's body during a first interval of time; and
(b) applying alternating voltages at a frequency between 50 kHz and 1 MHz between four secondary sets of one or more electrode elements positioned on four secondary non-overlapping regions on the subject's body during a second interval of time,
wherein each of the primary regions has an area, and wherein 0-20% of the area of each of the primary regions, taken alone, is overlapped by all of the secondary regions taken together, and
wherein the first interval of time and the second interval of time are non-overlapping.

14. The method of claim 13, wherein none of the secondary regions overlap any of the primary regions.

15. The method of claim 13, further comprising repeating steps (a) and (b) in an alternating sequence at least ten times.

16. The method of claim 13, wherein the second interval of time is separated from the first interval of time by less than 48 hours.

17. The method of claim 13, wherein the first interval of time is at least 12 hours and the second interval of time is at least 12 hours.

18. The method of claim 13, further comprising:
(c) prior to the first interval of time, positioning the primary set of one or more electrode elements on the primary regions;
(d) after the first interval of time, removing the primary set of one or more electrode elements from the subject's body;
(e) prior to the second interval of time, positioning the secondary set of one or more electrode elements on the secondary regions; and
(f) after the second interval of time, removing the secondary set of one or more electrode elements from the subject's body.

19. The method of claim 18, further comprising repeating steps (c), (a), (d), (e), (b), and (f), in that order, at least ten times.

20. The method of claim 19, wherein each of the primary regions and each of the secondary regions are located on the subject's abdomen.
